(19) United States
(12) Patent Application Publication
(10) Pub. No.: US 2026/0358320 A1
(43) Pub. Date: Nov. 12, 2026

(54) METHOD FOR LITHOLOGICAL CLASSIFICATION AND HIERARCHISATION OF BUILDING BLOCKS

(71) Applicant: Petróleo Brasileiro S.A. - Petrobras, Rio de Janeiro (BR)

(72) Inventor: Matheus Augusto ALVES CUGLIERI, Rio de Janeiro (BR)

(73) Assignee: Petróleo Brasileiro S.A. - Petrobras, Rio de Janeiro (BR)

(21) Appl. No.: 19/488,576

(22) PCT Filed: Jun. 7, 2024
(86) PCT No.: PCT/BR2024/050237

§ 371 Date: Nov. 26, 2025

(30) Foreign Application Priority Data
Jun. 9, 2023 (BR) 1020230113532

(51) Int. Cl. G01N 33/24; G01N 15/0227; G06V 10/764

(52) U.S. Cl. CPC G01N 33/24; G01N 15/0227; G06V 10/764

(57) ABSTRACT

Described herein are systems and methods for lithological classification of a rock based on the classification and hierarchization of constituent elements of the rock. A user defines a lithological classification system to be used. The user establishes the order of relevance of each constituent element of each member of the lithological classification system. The presence or absence of the constituent elements is then recognized in an analyzed petrographic image of the rock. The presence or absence of the constituent elements in the image is compared to the lithological classification system to determine a lithological classification of the rock in the image.

### CROSS-REFERENCE TO RELATED APPLICATIONS

This application is a U.S. national stage application under 35 U.S.C. § 371 of International Application No. PCT/BR2024/050237, filed Jun. 7, 2024, which claims priority of Brazilian Application No. 1020230113532, filed Jun. 9, 2023. The entire contents of each priority application are incorporated herein by reference.

### FIELD

The present invention falls within the field of technologies for lithological classification; that is, rock classification systems, more specifically, a lithological classification system based on the classification and hierarchization of the constituent elements.

### BACKGROUND

In order to provide a better understanding of the present invention, a brief explanation of the state of the art, as well as the existing problems, will be presented, providing a person skilled in the art with information to recognize the existing limiting aspects and an understanding on the technical features of the proposed solution to the problems.

Lithological classification is one of the main tools of petrographic analysis, with a microscopic approach being essential for greater accuracy. This task requires a high degree of specialization and extensive time of analysis to provide information on a microscopic scale regarding the structure, texture, and mineral phases that make up the lithology.

When performed manually, the analysis relies on the specialist's ability to recognize the constituents of the lithological texture and, based on the arrangement between these constituents and the relevance of each, a classification is proposed, related to a system of pre-determined members.

By “Mineral Phase” is meant “a petrographic constituent composed of its own external habit/geometry, internal structure, size/crystallinity, mineralogy and paragenetic relationships”. These are the “building blocks” of the solids in a lithological texture.

Among the attributes considered in the definition of “Mineral Phase”, two groups of characteristics can be distinguished: the first corresponds to the mineralogical composition, which is determined by the nature of the minerals that make up the rock, with no inference regarding its genetic history; the second, referred to as the “Constructive Element”, concerns the set of internal and external spatial features of the “building blocks”; that is, the materialization of the conditions under which the minerals formed and their relationships with adjacent features, which are relevant to the rock's genetic history.

Typically, the work of a petrographer is aimed at both discretizing the constituents of these two cited groups using specific petrographic criteria based on observation and the use of resources available in microscopic equipment, and articulating these information domains to achieve interpretations regarding the geological processes to which that rock was subjected, supporting inferences related to the most diverse purposes (genetic interpretation, quality of the reservoir, and so on.).

A rock classification system is a functional relationship between different members, designed to serve a specific context and with its own purposes. The classification process is based on the expert's ability to recognize the main constituents of the lithological texture (i.e., the “Constituent Elements” can be used as the key unit of this classification) and, in practice, to hierarchize and quantify them when necessary.

In other words, lithologies are classified according to the occurrence and arrangement of certain constituents. By arrangement is meant not only the frequency (in this instance, the percentage of area occupied by the constituent), but also the relevance of that constituent in the classification system. This is because certain constituents are more relevant than others; that is, they are more characteristic for defining certain members of the classification system.

This sense of importance is based on the expert's reasoning, who in addition to recognizing these constituents and often providing an estimated quantification, formulates the classification hypothesis based on this fact.

Certain lithologies require the Constituents Elements to be quantified, such as granular textures whose classifications depend on the presence and the number of grains of certain sizes, while others only require the arrangement of their occurrences.

Some automated lithological classification initiatives are based on general properties of petrographic images (distribution of RGB patterns of the pixels that form the image, for example), which does not necessarily reflect the attributes required for lithological classification, generating inaccurate results.

Some classification systems include members with diverse textural aspects resulting from variations in the amounts of certain constituents without, however, changing the lithological type. This results in lithologies with considerably distinct textural aspects.

With the advent of artificial intelligence tools, including clustering algorithms, this issue has become popular given the nature of these methods in separating and clustering groups exhibiting similar properties.

In many contexts, this approach does not provide effective solutions to the classification problem, since different lithologies may exhibit the same textural aspect in terms of the distribution of properties of the pixels. It is therefore interpreted that current methods do not address the issue of automated lithological classification within the petrographic scope, in the sense of adopting an essentially petrological method to solve the problem.

Therefore, according to the above issues, it is an object of the present invention to solve the aforementioned problems existing in the State of the Art, mainly related to the automated lithological classification that provides inaccurate results, seeking better reliability, optimizing the workflow, and improving yield, making the classification faster and more effective, since, by automating the process, a high degree of specialization and a long analysis time would no longer be required to achieve the information about the rock. In addition to contributing to more assertive and data-driven decisions in daily work routines.

### STATE OF THE ART

The search for prior arts related to the present invention has led to some documents that disclose subject matter that is within the technological field of the present invention; however, in these documents, one or more of the aforementioned technical problems were still identified.

Document CN111563445 refers to the classification and identification of rock lithology; in particular to a microscopic lithology identification method based on a convolutional neural network, which comprises the following steps: acquiring a plurality of rock slices microscopic images to be identified as a sample set; inputting the sample set into a trained convolutional neural network, and outputting classification information of the sample set, wherein a rock slice microscopic image set is obtained by adopting orthogonal polarized light or single polarized light or both of the orthogonal polarized light and the single polarized light, and the rock slice microscopic image set forms the sample set. According to the method, a computer is used for automatically obtaining feature description of the images through learning and automatically classifying the images. Labor cost and the learning cost are remarkably reduced, the lithology identification speed is greatly increased, and the method has the advantages that the oil and gas exploration and development benefits are improved efficiently and conveniently, and the development cost is reduced.

However, the method proposed in CN111563445 establishes that the algorithm used must recognize the similarities between the analyzed image and the library created. Such similarity is based on general properties of the images; for example, the distribution of color channels of pixels that make up the images. Therefore, the algorithm used will tend to classify the analyzed image according to its similarity to some specific group of images. Following the given example, such similarity would be related to a similar distribution of color channels. However, for such an approach to always yield satisfactory results, all rocks classified as a certain type (for example: all rock samples classified as “X”) should have the same characteristics or similar properties. However, in reality, this is not the case, since what defines a lithological classification is its structural composition (the constituent elements) rather than its general appearance. Sometimes the overall appearance can represent such structural composition, but this is not usually the case. The result of this, in terms of automated systems, is the inaccuracy of this type of automated classification; that is, the automated system provides an erroneous classification that does not reflect the reality of the rock being analyzed.

Document CN109655933 discloses an unconventional formation lithology identification method and system. The method comprises the steps that 1) a logging curve of a target area is pre-processed, and the pre-processed logging curve is used to automatically stratify unconventional formation intervals; 2) sensitive logging parameters of unconventional formation petrophysical facies are extracted from all logging parameters of a key well; 3) the petrophysical facies of each layer in the key well are divided, and based on different logging response characteristics of different petrophysical facies, the corresponding relationship between the petrophysical facies and geological facies is established; and 4) the logging parameters of an unconventional formation to be identified are extracted; the petrophysical facies of the unconventional formation are identified layer by layer; based on the established corresponding relationship between the petrophysical facies and the geological facies in step 3), the lithology of each layer is identified. According to the invention, the petrographic identification method has more precise identification and more accurate division than a geological logging lithology profile, can replace logging and lithology test, is not subject to regional limitations, and has obvious social and economic benefits.

However, the method proposed in CN109655933A addresses other types of data (profile curves). It is a method that addresses indirect data (profile curves result from the response of rocks to different stimuli—passage of electric current, acoustic waves—or the reading of emanations from these rocks—radioactivity, for example), and by combining this information, it proposes an estimated lithological classification. As already discussed, it addresses other types of data, both in nature and in study scale.

Document CN108035710 is guided by existing geological knowledge, according to complex geological characteristics of deep rock in a target area; types of lithofacies are divided, lithofacies division scheme is obtained; a method of logging response radar map is used to merge the lithofacies which cannot be distinguished by logging information. The lithofacies division scheme is obtained; sensitive logging parameters are selected, a rendezvous diagram is established, a new logging facies is obtained by the method of data mining, and the new logging facies is combined with the actual needs of deep oil and gas reservoir evaluation, geofacies division scheme and division standards are established. Based on the geological knowledge and the logging data mining, the method of dividing deep geofacies can be extended to different geological conditions and different depths of application environment, is especially suitable for the problem of the division of deep lithofacies.

However, the method of CN108035710 uses indirect data (profiles), rather than advanced petrographic concepts for lithological classification.

Document US2021215590 describes an intelligent lithology identification system and method based on images and spectrum technology. The intelligent lithology identification system includes a rock shape analysis system, an image identification system, a sample processing system, a spectrum analysis system, and a central analysis and control system; wherein the central analysis and control system determines the final lithology of a sample according to the rock identification results from the image identification system and the analysis results from the spectrum analysis system. The technical solution further identifies the content and type of minerals by using spectrum technology, integrates and analyzes the results of spectrum analysis and image identification, and finally gives the lithology of the rock, which greatly improves the accuracy of lithology identification.

Document US2020005013AA describes a workflow for rock sample image processing is established to determine geometrical parameters of rock texture. Individual pores and grains are identified using Hoshen-Kopelman multi-cluster labeling algorithm and watershedding technique. Separated elements are fitted with ellipses and pore/grain size distributions, aspect ratios and orientations of fitting ellipses are obtained. Such information is especially valuable in interpretation and forward modeling of dielectric responses using textural models. Rock sample images by high-resolution confocal microscopy are selected to test the workflow and results are analyzed.

However, the method of US2020005013AA appears to deal with geometries, both of the pores and the solid phase, but only quantitatively (radius, diameter, axis orientation, etc.), but not qualitatively, as with the constituent elements.

In summary, a person skilled in the art in possession of CN109655933, which provides a system for classifying rock lithology based on the relationship between petrophysical facies and geological facies; that is, predetermined parameters, upon encountering document US2021215590, which analyzes the rock from images obtained of textures in order to achieve the lithological classification, and US2020005013, which identifies the rock lithology by identifying geometric parameters of the rock texture, would not be able to classify and hierarchize textures (Constituent Elements) and the geometric parameters thereof that make up the rock microstructure in order to classify the lithology of that rock. This is because the hierarchization (in the sense of the relevance of each Constituent Element for the lithological classification) is carried out before the recognition of the constituent elements in the petrographic thin section photograph, and said knowledge acquired by the already cited mechanisms would be implemented in the proposed method through the development of the cited relationships. In other words, the method comprises the mechanisms and relationships between processes and decision-making to automate the lithological classification process.

Therefore, none of the methods presented in the prior art documents proposes the method according to the present invention, which will be presented throughout the present specification.

### SUMMARY

The method described in the present invention is based on reproducing the petrographic work carried out by an expert, through automated processes, and is based on four steps.

In step I, the classification system to be used is defined, with all its members, each of the members having the aforementioned Constituent Elements.

In step II, the order of relevance of each Constituent Element is established to arrive at the classification of each member of the classification system, where the most characteristic Constituent Elements are those that exhibit an exclusive relationship with a certain member of the classification system recited in step I.

In step III, the presence, or absence, of the existing Constituent Elements is identified in the petrographic image being examined.

Finally, in step IV, the arrangement of presences/absences of the Constituent Elements will be compared with the relationships established in step II to achieve a proposed classification in relation to the members established in step I.

Therefore, the proposed method provides a more precise lithological classification, since it is based on the identification of the constituents of lithological textures (Constituent Elements), instead of properties of the image, which are amenable to various possible variations, both related to the lithological context and to the context of thin section preparation and photodocumentation.

Furthermore, the proposed classification is a result of the step of automated recognition of constituents and the application of this content to a set of classification instructions fed with the required information to generate the proposed classifications. This makes it easy for the user to change the classification system concepts, adapting the results to their needs, without having to make adjustments to the information acquisition process.

### BRIEF DESCRIPTION OF THE FIGURES

In order to provide a complete understanding of the object of the present invention, reference is made to the accompanying figures described below.

FIG. 1 illustrates the definition of Constituent Element and Mineral Phase.

FIG. 2 shows an example of the application of the proposed method.

### DETAILED DESCRIPTION

The method described in the present invention is based on reproducing the petrographic work carried out by an expert, through automated processes. Thus, the classification process is based on the expert's ability to recognize the main constituents of the lithological texture (i.e., the “Constituent Elements” which are used as the key unit of this classification) and, in practice, to hierarchize and quantify them.

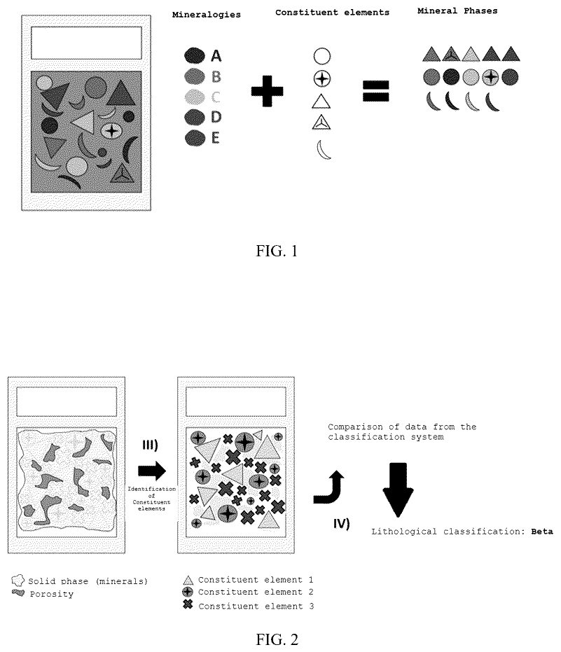

FIG. 1 illustrates the definition of Constituent Element and Mineral Phase. In a thin section (schematics on the left of the composition), two different types of solid-phase “information” can be identified for classification purposes: the mineralogical composition and its distribution profile within the solid volume/area.

By “Constituent Element” is meant the manner in which different mineralogies will be distributed within the sample. They have their own characteristics in terms of shape, structure, size, and relationships with the surroundings. Once the mineralogical information is combined with this knowledge of the solid framework, the more complete concept of “Mineral Phase” is formed, which can be interpreted as the building block of the solid framework, susceptible to the depositional and diagenetic history of the rock.

Therefore, the work of a petrographer is aimed at both discretizing the constituents of these two cited groups using specific petrographic criteria based on observation and the use of resources available in microscopic equipment, and articulating these information domains to achieve interpretations regarding the geological processes to which that rock was subjected, supporting inferences related to the most diverse purposes (genetic interpretation, quality of the reservoirs, and so on.).

Basically, when the expert performs this analysis, three main domains of information are sought: definition of porosity (empty space), that is, the “voids”; the mineralogical composition of the rock, based on the identification of different minerals using optical criteria (color, interference color, relief, type and angle of extinction, and sensitivity to certain chemicals on the mineral surface-all of which are optical properties resulting from the interaction between the mineral structure and the passage of the beam of transmitted light); and the manner by which these mineralogies are distributed in terms of the rock's “structural elements”, herein referred to as “Constituent Elements.”

Therefore, it should be noted that defining the minerals that make up a rock does not provide a classification for it. Such mineralogical characteristic is one of the features that can aid in lithological classification, but it is not the information that determines the classification.

On the other hand, the “Constituent Element” is characterized by a defined structure, with its own external and internal characteristics, and specific relationships with other elements, which are defined as “paragenetic relationships”. Identification of these constituent elements present in the thin section of the rock that makes it possible to classify that rock according to a classification system.

Thus, what defines whether a rock (represented by the analyzed thin section) is classified as “X” or “Y” is, more importantly, the types of Constituent Elements present therein and, less importantly, the number of elements. Therefore, recognizing the Constituent Elements is essential for classification.

The proposed method aims to replicate the work of an expert in order to automatically classify and hierarchize the Constituent Elements, thereby expediting the task and eliminating the need for manual lithological classification.

Step I of the method consists of defining the classification system(s) to be used, with all its members. In other words, in step I the user predefines the relationships between the members.

The members correspond to the sets of lithological classifications predefined by the user; that is, each member represents a lithological classification, which may correspond to a rock type identified at the end of the method.

In step II of the method, the user establishes the order of relevance of each Constituent Element to arrive at the classification of each member of the classification system. The most characteristic Constituent Elements are those that have an exclusive relationship with a particular member of the classification system, mentioned in step I;

Therefore, the user defines a lithology, a description of that lithology, and the relationships of the Constituent Elements with said predetermined lithology. In other words, it describes the constituent elements of each rock classification term, the exclusivity of the constituent elements for certain rock classification terms, etc.

Table 1 below shows an exemplary classification system to be defined for applying the method:

TABLE 1
Exemplary classification system

| | | Constituent elements | |
|---|---|---|---|
| Lithology | Description | + exclusive | −exclusive |
| Alpha | Description of petrographic | [symbol] | [symbol] |
| | characteristics of alpha | | |
| Beta | Description of petrographic | [symbol] | [symbol] |
| | characteristics of beta | | |
| Charlie | Description of petrographic | [symbol] | |
| | characteristics of charlie | | |
| Delta | Description of petrographic | [symbol] | [symbol] |
| | characteristics of delta | | |

For each lithology in the system (first column, on the left), there is an (optional) description and a list of the constituent elements that comprise it. It should be noted that certain constituent elements are “exclusive” to specific lithologies; that is, they occur in a single lithology, making them diagnostic of that lithology. Other elements are common to more than one member of the classification system; therefore, their relevance to lithological classification is low.

Therefore, the method being presented is a “generic” step-by-step process, applicable to any context, the user being responsible for describing the peculiarities inherent to the adopted classification system and generating the set of instructions for recognizing the Constituent Elements.

Steps I and II are part of the “contextualization” of the set of instructions of the method, which can be understood as a reproduction of the expert's knowledge to perform the task (Table 1). Both steps aim to provide the basis to enable the processing of the collected data and the synthesis of information related to lithological classification automatically.

In step III of the method, the system recognizes in the analyzed petrographic image, the presence, or not, of the existing Constituent Elements.

Step III deals exclusively with the ability to recognize the Constituent Elements; that is, the ability of the sets of instruction to identify the building blocks of lithological textures (FIG. 2). In this step, the greater the accuracy in recognizing the Constituent Elements, the better the results obtained. This gain in precision mimics the experience and knowledge of an expert.

Step III is the step of the described method in which the task of the expert will be reproduced, since, in this step, the system will identify the three main information domains, which have already been previously recited:
The porosity, the aforementioned empty spaces, the “voids,” which, in the case of thin sections, are usually filled with blue-colored resin;
the mineralogical composition of the rock, excluding the porosity spaces; and
the Constituent Elements; that is, how these mineralogies are distributed in terms of the “structural elements” of the rock.

Therefore, in order to obtain a good analysis of petrographic thin sections, the images preferably have sufficient resolution (pixel size) and optical magnification, which can be translated as “optical zoom level,” for recognition of the constituent elements.

If a thin section image has a pixel size larger than the size of the Constituent Elements, this prevents the method from being applied, since said Constituent Elements would not be represented at that resolution.

However, whether this image is obtained through a specific camera, manually (by the user acquiring the photos) or automatically (software operating a microscope-camera setup), there is no difference, since how these images are obtained is irrelevant to the method described herein.

Finally, in step IV, the arrangement of presences/absences of the Constituent Elements will be compared with the relationships established in step II to achieve a proposed classification in relation to the members established in step I.

Thus, step IV reproduces the reasoning that the expert develops to classify the lithology within the classification system used (FIG. 2).

In other words, after step IV, the Constituent Elements are identified in the petrographic thin section image, and an order of “relevance” of these Constituent Elements present in the petrographic thin section is applied to classify the rock.

FIG. 2 shows an example of the application of the method proposed in the present invention. On the left, there is a thin section diagram prior to the identification of the Constituent Elements (step III of the method). After identifying these elements, the solid phase present is subdivided into the different Constituent Elements identified. This catalog of discretized content is compared with the classification table populated in steps I and II, in order to find the best member of the classification system corresponding to the identified content (step IV).

Based on the examples below, the importance of correctly identifying and classifying the Constituent Elements becomes clear, so that the classification and hierarchical system of the Constituent Elements is efficient.

The first example cited here is the case of a rock designated as “X”, composed of elements “a”, “b”, and “c”. If, when assessing a photograph of a thin section, elements “b” and “c” are identified, but not element “a”, classification this rock will not be “X”, but rather another term that is characterized only by elements “b” and “c”.

The second example cited here is a rock designated “Z”, composed of elements “1”, “d”, and “s”. The identification of “1”, regardless of the amount, is sufficient to classify the rock as “Z”, since “1” is a unique Constituent Element of rock “Z”.

In other words, identifying an element “1”, regardless of its amount, is sufficient to classify a rock if “1” is an exclusive Constituent Element of that rock.

Thus, the method of lithological classification using a classification and hierarchization system of the constituent elements is defined by:
I) defining at least one lithological classification system to be used, with all its members;
II) establishing the order of relevance of each Constituent Element of each member of the classification system recited in step I;
III) recognizing, in a petrographic image, the presence, or not, of the existing Constituent Elements; and
IV) comparing the arrangement of presences/absences of the Constituent Elements with the relationships established in step II in relation to the members established in step I;
   wherein, by comparing the arrangement of the presence or absence of the Constituent Elements with the relationships established in step II with respect to the members defined in step I, the lithological classification of the rock is carried out by determining its constituents. It is reiterated that each member of the classification system represents a lithological classification, which may be associated with a rock classified at the end of the method.

The aforementioned classification system identifies three main information domains: porosity, mineralogical composition of the rock, and constituent elements.

An image of a petrographic thin section analyzed in step III preferably has sufficient resolution (pixel size) and optical magnification (optical zoom level) to recognize the constituent elements (on the scale of micrometers to a few millimeters).

The petrographic thin section image can preferably be achieved using a camera operated by a set of instructions that automatically define the microscope settings.

Therefore, the proposed method provides a more precise lithological classification, since it is based on the identification of the constituents of lithological textures (Constituent Elements), instead of properties of the image, which are amenable to various possible variations, both related to the lithological context and to the context of thin section preparation and photodocumentation.

Furthermore, the proposed classification is a result of the step of automated recognition of constituents and the application of this content to a set of classification instructions of the method fed with the required information to generate the proposed classifications. This makes it easy for the user to change the classification system concepts, adapting the results to their needs, without having to make adjustments to the information acquisition process.

Those skilled in the art will value the knowledge presented herein and may reproduce the invention in the disclosed embodiments and other variants, as covered by the scope of the appended claims.

1. A lithological classification method, comprising:
I) defining at least one lithological classification system to be used, with all its members;
II) establishing an order of relevance of each Constituent Element of each member of the classification system recited in step I;
III) recognizing, in a petrographic image, a presence, or not, of the existing Constituent Elements; and
IV) comparing an arrangement of presences/absences of the Constituent Elements with relationships established in step II in relation to the members established in step I;
wherein, by comparing the arrangement of the presence or absence of the Constituent Elements with the relationships established in step II with respect to the members defined in step I, lithological classification of the rock is carried out by determining its constituent elements.

2. The lithological classification method of claim 1, wherein each member of the classification system represents a lithological classification, which may be associated with a rock classified at an end of the method.

3. The lithological classification method of claim 1, wherein the lithological classification system identifies three main information domains: porosity, mineralogical composition of the rock, and constituent elements.

4. The lithological classification method of claim 1, wherein a petrographic thin section image analyzed in step III preferably has sufficient resolution (pixel size) and optical magnification (optical zoom level) to recognize the constituent elements (on the order of micrometers to a few millimeters).

5. The lithological classification method of claim 1, wherein the petrographic thin section image can preferably be achieved using a camera operated by a set of instructions that automatically define [the] microscope settings.

6. The lithological classification method of claim 1, wherein after identifying the Constituent Elements in the thin section image, in step IV, the order of relevance of Step III of said Constituent Elements present in the petrographic thin section are applied.

7. The lithological classification method of claim 1, wherein identifying an element “1”, regardless of its amount, is sufficient to classify a rock if “1” is an exclusive Constituent Element of that rock.
